(19) United States
(12) Patent Application Publication
(10) Pub. No.: US 2003/0163342 A1
(43) Pub. Date: Aug. 28, 2003

(54) Method for defraying storage costs

(76) Inventor: Jeffrey Charles Trewella, Kennett Square, PA (US)

Correspondence Address: Jeffrey C. Trewella, c/o Innovative Solution$ Inc., 283 Hickory Drive, Kennett Square, PA, 19348, US

(21) Appl. No.: 10/084,523

(22) Filed: Feb. 28, 2002

(51) Int. Cl. C12N 5/00; G06F 17/60

(52) U.S. Cl. 705/1; 435/325

(57) ABSTRACT

A method for defraying the cost of a storage service for a divisible medical material comprising dividing said material into at least two portions, selling a first portion of said material and using the proceeds from said sale to defray the cost of storing a second portion of said material.

### BACKGROUND OF THE INVENTION

Medical science has recently focused a great deal of attention and research on the value of human stem cells for treating various maladies. Stem cells have been demonstrated to have the capacity to differentiate into more specialized cells, raising the possibility of repairing nerve damage or even growing new organs from the cells themselves. These cells are found naturally in a number of locations in the human body, including in bone marrow, embryos and umbilical cord blood.

However, due to ethical concerns, human stem cell research has been greatly limited by reduced federal funding and by federal policies that limit funding for the use of embryonic stem cells which might be derived from embryonic sources not in existence as of today.

Fortunately, other sources of stem cells exist, especially those present in umbilical cord blood derived from the placenta and umbilical cord of the mothers of newborns. This source of new stem cells would seem limitless, and promises a continuing supply of stem cells for future medical research.

In anticipation of medical breakthroughs in repair and replacement of damaged body parts with stem cells, many parents are now seeking to recover the stem cells present in their newborn's umbilical cord blood and store that medical material on a long term basis, for the benefit of their child in the event of an accident or a chronic illness in the future. It is well known that a person's own tissue, for example blood, if stored and used later, will be unlikely to be rejected by their body's natural defenses, in contrast to rejection problems due to transplantation of tissues from another person.

A business has developed to provide long term storage facilities for umbilical cord blood, and thereby stem cells, wherein a newborn's umbilical cord blood is recovered with the permission of the parent(s) and placed into long term cryogenic storage. However, the recovery and storage operations are costly, which prevents many families of lesser means from securing the possible benefits of having their children's stem cells available at some point in the future.

It would be desirable to help defray the costs of recovery and storage of various medical materials, such as umbilical cord stem cells, which would permit greater access to these services to the general public.

Likewise, it would be desirable to enhance the availability of human stem cells to medical researchers.

### BRIEF SUMMARY OF THE INVENTION

I have invented a business method which can help solve the problems stated above, i.e. providing access to long term storage of medical materials for individuals, as well as increasing the supply of such materials for medical research.

One embodiment of the present invention is a method for defraying the cost of a storage service for a divisible medical material comprising dividing said material into at least two portions, selling or donating a first portion of said material and using the proceeds from said sale or tax credits from said donation to defray the cost of storing a second portion of said material.

Another embodiment of the present invention is a method for defraying the cost of a storage service for umbilical cord blood stem cells comprising dividing said umbilical cord blood into at least two portions, selling or donating a first portion of said umbilical cord blood and using the proceeds from said sale or tax credits from said donation to defray the costs of storing a second portion of said umbilical cord blood.

### DETAILED DESCRIPTION OF THE INVENTION

According to the invention, the cost of long term storage, and preferably the cost of recovery of divisible medical materials, such as umbilical cord blood, is defrayed by permitting the division of the material into at least two portions, storing one portion and selling the other portion to medical researchers and the like, and using the proceeds from the sale to reduce or eliminate the costs of storage and/or recovery.

Such a method provides a benefit to children of parents or other guardians of lesser means, who might not be able to afford recovery and/or long term storage of their child's umbilical cord blood. By permitting the storing facility to divide the recovered material into at least two portions, it is possible to retain one portion in long term storage, while the proceeds from the sale or donation of the remaining portion(s) can be used, at least in part, to pay at least a portion of the costs of storage for the parents. A financial benefit may be derived in the form of tax credits in exchange for a donation of a portion of the medical material to a charity or the like.

A further benefit is obtained by increasing the supply of medical materials, in this case umbilical cord blood containing stem cells, which are available for medical research or even treatment.

As those skilled in the art can imagine, the method of the present invention can be applied to other divisible medical materials, such as for example liver tissue. Recent research on liver tissue has indicated that it is renewable within an individual, and perhaps could be harvested and replicated in vitro for later transplantation into a person in need of a liver transplant.

While the essential steps of the present invention are dividing the medical material into at least two portions; and then benefiting financially from at least one portion of the divided medical material, additional steps may be conducted in order to practice the present invention. Initially, the divisible medical material must be collected or recovered, which step can be conducted either by a person practicing the present invention, or by an individual in a delivery room or hospital operating room who may be under contract to collect and deliver the medical material to one who wishes to practice the invention.

In one preferred embodiment, umbilical cord blood is recovered from the umbilical cord of a newborn infant by either aspiration with a syringe or by draining the blood from the umbilical cord into a container, using gravity and/or pressure on the umbilical cord.

In some instances it may be desirable to concentrate the umbilical cord blood, either prior to or subsequent to the dividing step. Likewise, it may be desirable to dilute and/or purify the umbilical cord blood, either prior to or subsequent to the dividing step. Concentration, dilution and purification can be conducted according to techniques well-known in the art.

Additionally, it may be possible to amplify the sample; in the case of stem cells in umbilical cord blood, to initiate or permit reproduction by the cells in order to obtain a greater number of cells with which to work. Obviously, the amplification step can be conducted either prior to or subsequent to division of the sample.

After division of the medical material into two or more portions, one portion is placed into an appropriate storage facility, for example a cryogenic storage tank, for long term storage for the donor. A second portion is then sold to a medical research facility, for example, the National Institutes of Health, medical research hospitals, charitable stem cell banks, or the like, the proceeds from the sale or the tax credits from the donation of that second portion being subtracted from the storage cost charged the donor, or can be rebated to the donor.

Those skilled in the art will recognize that the present invention can be modified in a number of ways, such as for example leasing the second portion of the material rather than selling it, without deviating from the practice of the present invention.

Those skilled in the art will also recognize that the financial and medical benefit from one portion of the divisible medical sample can be different from those of the other portions. For example, angiogenesis is the process by which new blood vessels are formed by pre-existing vessels. Angiogenesis is an important event in both normal physiological and pathological conditions. Several current antitumor drugs target angiogenesis pathways to modulate tumor growth. Thrombospondin is a natural 450,000 molecular weight protein that is secreted by blood platelets in response to physiological activators including collagen and thrombin (Lawer, J. Blood (1986) 67:112-123). Results suggest that thrombospondin can have both an inhibiting and a stimulating effect on angiogenesis (Pazouki S, et. al. Bochem Soc Trans (1996) 24:368S). In the present invention, the financial and medical benefit from one portion of the divisible medical material can be derived from the therapeutic regulation of angiogenesis derived from thrombospondin isolated from platelets present in a sample, and the benefit and use of another portion of the sample be derived from stem cells.

What is claimed is:

1. A method for defraying the cost of a collection or storage service for a divisible medical material comprising dividing said material into at least two portions, selling or donating a first portion of said material and using the proceeds from said sale or tax credits from said donation to defray the cost of collecting the material or storing a second portion of said material.

2. The method according to claim 1, further comprising recovering said material from a donor.

3. The method according to claim 1, further comprising concentrating said material.

4. The method according to claim 3, wherein said concentrating occurs prior to said dividing.

5. The method according to claim 1, further comprising diluting said material.

6. The method according to claim 5, wherein said diluting occurs prior to said dividing.

7. The method according to claim 1, further comprising purifying said material.

8. The method according to claim 7, wherein said purifying occurs prior to said dividing.

9. The method according to claim 1, further comprising amplifying said material.

10. The method according to claim 9, wherein said amplifying occurs prior to said dividing.

11. A method for defraying the cost of a storage service for umbilical cord blood stem cells comprising dividing said umbilical cord blood into at least two portions, selling or donating a first portion of said umbilical cord blood and using the proceeds from said sale or tax credits from said donation to defray the costs of storing a second portion of said umbilical cord blood.

12. The method according to claim 11, further comprising recovering said umbilical cord blood from a donor.

13. The method according to claim 11, further comprising concentrating said umbilical cord blood.

14. The method according to claim 13, wherein said concentrating occurs prior to said dividing.

15. The method according to claim 11, further comprising diluting said umbilical cord blood.

16. The method according to claim 15, wherein said diluting occurs prior to said dividing.

17. The method according to claim 11, further comprising purifying said umbilical cord blood.

18. The method according to claim 17, wherein said purifying occurs prior to said dividing.

19. The method according to claim 11, further comprising amplifying said umbilical cord blood.

20. The method according to claim 19, wherein said amplifying occurs prior to said dividing.

21. The method according to claim 11, further comprising stabilizing said umbilical cord blood.

22. The method according to claim 21, wherein said stabilizing occurs prior to said dividing.
